(19) United States
(12) Patent Application Publication
(10) Pub. No.: US 2025/0302487 A1
(43) Pub. Date: Oct. 2, 2025

(54) ORTHOPEDIC CUTTING APPARATUS AND GUIDE FEATURES

(71) Applicant: Arthrex, Inc., Naples, FL (US)

(72) Inventors: Reinhold Schmieding, Naples, FL (US); Nick Metcalfe, Bonita Springs, FL (US); Matthew Provencher, Edwards, CO (US)

(73) Assignee: Arthrex, Inc., Naples, FL (US)

(21) Appl. No.: 19/083,984

(22) Filed: Mar. 19, 2025

Related U.S. Application Data
(60) Provisional application No. 63/569,929, filed on Mar. 26, 2024.
(60) Provisional application No. 63/687,009, filed on Aug. 26, 2024.

(51) Int. Cl. A61B 17/15; A61B 17/17

(52) U.S. Cl. CPC A61B 17/151; A61B 17/1732

(57) ABSTRACT

A surgical cutting apparatus includes a cutting tool with a cutting head extending along a longitudinal axis to an acting end portion. A cutting guide includes an engagement surface configured to engage a first side of a bone and forms an opening extending from a guide surface. The guide surface forms a guide contour offset from and aligned with a positive contour of a second side of the bone. A depth guide is connected with the cutting tool and defines a cutting depth along the longitudinal axis, wherein the depth guide engages the guide surface along the opening and the cutting depth is adjusted along the opening by the engagement of the depth guide with the guide contour.

### CROSS-REFERENCE TO RELATED APPLICATIONS

This application claims priority under 35 U.S.C. § 119(e) and the benefit of U.S. Provisional Application Nos. 63/569,929 entitled ORTHOPEDIC CUTTING APPARATUS AND GUIDE FEATURES, filed on Mar. 26, 2024, by Schmieding et al., and 63/687,009 entitled ORTHOPEDIC CUTTING APPARATUS AND GUIDE FEATURES, filed on Aug. 26, 2024, by Schmieding et al., the entire disclosures of which are incorporated herein by reference.

### BACKGROUND

The present disclosure generally relates to a surgical cutting apparatus with various features that may assist in guiding the removal of patient tissue for various surgical procedures. In particular, the disclosure provides for various detailed examples including cutting guides that may provide depth stops defined by anatomical features of patients for osteotomies and similar procedures. As demonstrated in various detailed examples that follow, the guide features may be particularly advantageous for facilitating procedures that require tissue removal to a blind depth extending into a bone or similar anatomical structure.

### SUMMARY

Surgical procedures may benefit from advanced planning procedures, which may provide patient-specific information allowing surgeons to prepare and successfully correct various orthopedic injuries and disorders. In particular, the disclosure provides for a surgical cutting apparatus that may include a cutting guide having a guide surface forming one or more guide contours. The guide contours may be defined by one or more interior features or transitions within a bone or, more generally, within the anatomy of a patient. In various implementations, the cutting guide may include an engagement surface that is directed toward a first side of a bone and positioned to align the cutting guide with the patient. Once aligned, an opening of the cutting guide is arranged such that a cutting path of the cutting guide is positioned over a target region for performing an osteotomy. During a surgical procedure, a cutting tool may be deployed within the opening formed by the cutting guide, such that the resulting tissue removal associated with the osteotomy is aligned with the cutting path formed by the cutting guide. In this way, the cutting apparatus may provide for accurate alignment of the osteotomy with the anatomical features of the patient.

In various implementations, the guide contour formed by the guide surface of the cutting guide may be offset from and aligned with a positive contour on a second side of the bone of the patient. The positive contour may correspond to a blind depth profile formed along a feature or interior transition of the bone of the patient along the cutting path. In operation, a depth guide in connection with the cutting tool may be positioned within the opening of the cutting guide and in engagement with the guide surface along the opening, such that the depth of the cut along the cutting path varies in correspondence with a positive contour of the feature or interior transition of the bone. In this way, translation of the cutting tool within the opening of the cutting guide and along the cutting path may result in a channel formed through the bone with a depth that varies in correspondence with the features or transitions hidden in a volume of the bone of the patient.

In various implementations, the depth of cut of the cutting tool as defined by the guide contour of the cutting guide may be aligned with an interior transition of the bone of a patient from a first hardness to a second hardness. For example, the depth associated with the guide contour along the cutting path may vary to correspond with a transition of the bone from a softer cancellous bone tissue to a harder cortical bone tissue. In this configuration, the engagement of the cutting tool with the cutting guide may result in the removal of cancellous bone within an interior of the bone of the patient while retaining or preventing engagement of the cutting tool with cortical bone on the second side of the bone opposite the first side to which the cutting guide is engaged. Additionally, in various implementations, the operation of the surgical cutting apparatus and the cutting guide may be implemented in coordination with one or more cutting tools that may include different cutting heads to improve accuracy of the cut in a resulting osteotomy. Accordingly, the disclosure may provide for various devices and methods that may assist in the operation of a surgical cutting apparatus.

These and other features, objects and advantages of the present disclosure will become apparent upon reading the following description thereof together with reference to the accompanying drawings.

### BRIEF DESCRIPTION OF THE DRAWINGS

FIG. 1 is a projected environmental view of a surgical cutting apparatus exemplified in performing a high tistitial osteotomy;

FIG. 2A is top plan view demonstrating a proximal end of a bone demonstrating a transition between cancellous bone and cortical bone;

FIG. 2B is a top plan view of the bone demonstrated in FIG. 2A, further demonstrating the position of a surgical cutting guide including a guide contour of a depth guide;

FIG. 2C is a top plan view of the bone demonstrated in FIGS. 2A and 2B, further demonstrating a blind cutting operation for an osteotomy performed with the cutting guide;

FIG. 3A is a process diagram demonstrating cutting tools, including a plurality of cutting heads for performing a cutting method associated with a depth guide;

FIG. 3B is a side profile view demonstrating a plurality of cutting heads, including dull or blunt tips for utilization with a cutting guide;

FIG. 4A is a side profile view of a cutting tool demonstrating an exemplary depth guide;

FIG. 4B is a side profile view of a cutting tool demonstrating an exemplary depth guide;

FIG. 4C is a side profile view of a cutting tool demonstrating an exemplary depth guide;

FIG. 5 is a flow chart demonstrating a method of implementing a cutting guide for performing an orthopedic correction procedure;

FIG. 6 is a block diagram demonstrating a surgical console and controller for operating a cutting tool;

FIG. 7A is a side perspective view of a cutting tool comprising a plurality of cutting heads;

FIG. 7B is a projected view of the cutting tool demonstrated in FIG. 7A implementing a resection procedure;

FIG. 7C is a side profile view of a second cutting head of the cutting tool of FIG.7A forming an extended entry passage through a cortical bone;

FIG. 7D is a perspective view of the cutting tool demonstrated in FIG. 7C;

FIG. 8A is a side profile view of the cutting tool comprising a force-detection feature in an extended or unloaded position;

FIG. 8B is a side profile view of the cutting tool comprising a force-detection feature in a compressed or loaded position;

FIG. 9A is a side profile view of the cutting tool comprising a pressure release feature;

FIG. 9B is a side profile view of the cutting tool comprising a pressure release feature;

FIG. 9C is a top view and projected view of the cutting tool comprising;

FIG. 10A is a cutting tool comprising a distal retraction stop oriented in a stowed position;

FIG. 10B is a cutting tool comprising a distal retraction stop oriented in a deployed position;

FIG. 10C is a side cross-sectional view of the cutting tool demonstrated in FIGS. 9A and 9B implemented in an exemplary surgical procedure;

FIG. 11 is a side view of a cutting tool comprising a distal retraction stop; and

FIG. 12 is a side view of a cutting tool comprising a distal retraction stop.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

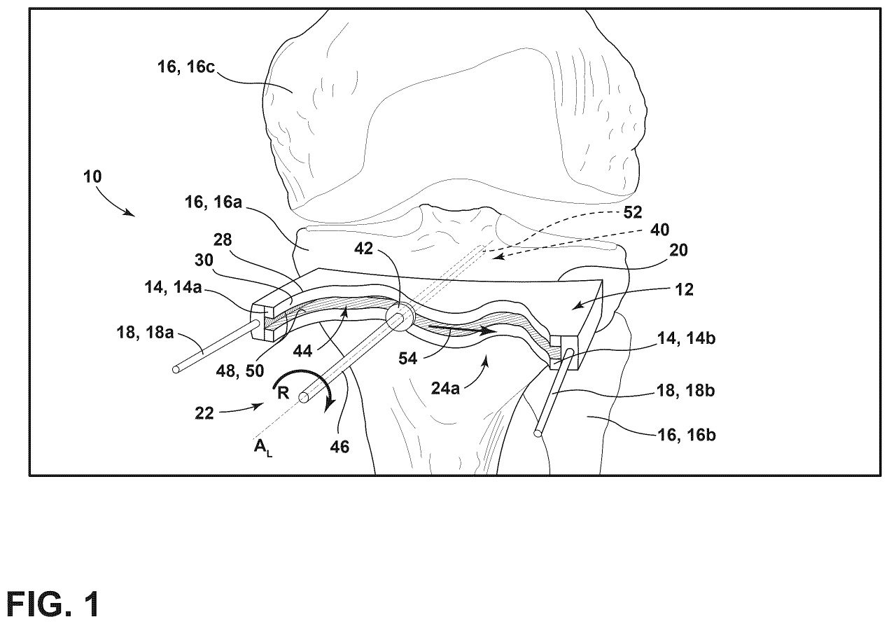

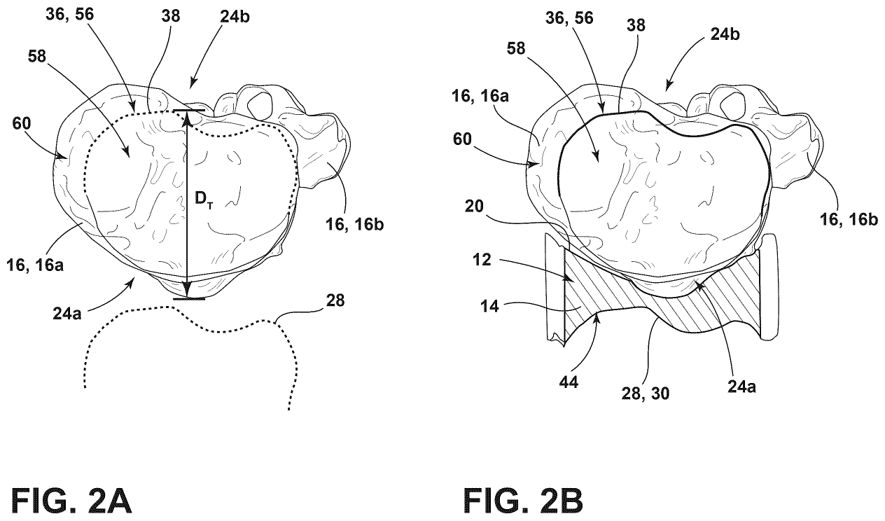

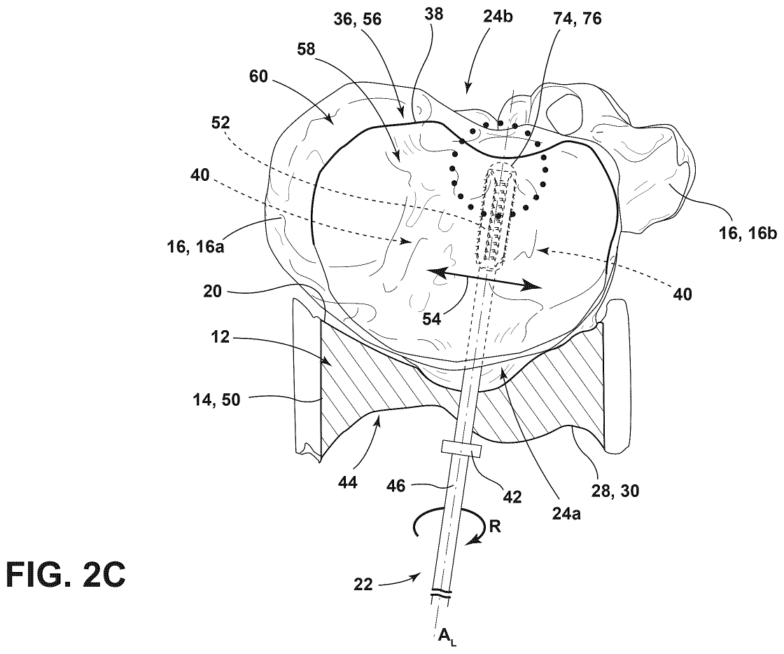

In various implementations, the disclosure may provide for a surgical cutting apparatus 10 and corresponding methods of operation that may improve and/or facilitate a variety of orthopedic procedures. Referring to FIGS. 1, 2A, 2B, and 2C, an exemplary surgical cutting apparatus 10 in accordance with the disclosure is shown performing an osteotomy. As shown, the procedure demonstrated in FIGS. 1 and 2 may correspond to a high tibial osteotomy. However, it shall be understood the apparatus 10 may be implemented for a variety of orthopedic procedures. In general, the surgical cutting apparatus may facilitate the correction or repair of a variety of orthopedic disorders or injuries.

In various implementations, the cutting apparatus 10 may include a cutting guide 12 comprising one or more guide plates 14 configured to be aligned with a bone 16 or similar structure of a patient. As demonstrated, the exemplary bone 16 may correspond to a proximal portion of a tibia 16a demonstrated in combination with fibula 16b and a distal end of a femur 16c. The alignment of the cutting guide 12 with the bone 16 may be achieved by affixing or otherwise positioning one or more alignment features 18 relative to the bone 16. In the example shown, the alignment features include a first pin 18a and a second pin 18b. However, in various implementations, the alignment features 18 may include one or more alignment surfaces that may extend over an engagement surface 20 directed toward a first surface 22 or first side of the bone 16. In some implementations, the engagement surface 20 may be implemented as a patient-specific alignment surface that may conform to the shape, contour, and/or proportions of the first surface 22 of the bone. Accordingly, the cutting apparatus may be implemented in a variety of standard or custom configurations to suit a procedure and/or a preference of a user.

As demonstrated in FIG. 1, the cutting guide is positioned such that the engagement surface 20 is positioned proximate to or in contact with a first surface 22 of the bone 16. As further discussed in the detailed examples that follow, the one or more guide plates 14 of the cutting guide 12 may be utilized to align a cutting tool 22 with the first surface 22 of the bone 16 to perform a blind cut partially through the bone 16 toward a second surface 24b opposing the first surface 24a. A blind target depth D_{T} of the cutting tool 22 through the bone 16 may controlled and limited by a guide profile 28 extending along a guide surface 30. In operation, a depth guide 32 of the cutting tool 22 may engage the guide surface 30 and extend into the bone 16 to a predetermined or metered cutting depth D_{C} that may limit the resulting tissue removal to align with the guide contour 28 of the guide surface 30 offset over the cutting depth D_{C}. As described in various examples that follow, these and other features of the cutting guide 12 may improve the efficiency and accuracy of various procedures, particularly those that may require extending the cutting tool 22 to a blind depth D.

As best demonstrated in FIGS. 2A and 2B, the guide profile 28 that defines the contour shape of the surface 30 may be aligned with an offset from an interior feature 36 or characteristic within an interior of the bone 16. For example, based on the interior feature 36 or characteristic of the bone 16, an interior cutting profile 38 may be defined. As demonstrated in FIG. 2A, the guide profile 28 of the guide surface 30 may be aligned with and offset from the interior cutting profile 38 over the target depth D_{T}. As shown in FIG. 2C, the guide surface 30 may conform to the guide profile 28, such that a longitudinal axis A_{L} of the cutting tool 22 may extend along the one or more guide plates 14 and into the bone 16 to a fixed cutting depth D_{C} controlled via a depth stop 42. In this configuration, a lateral movement of the cutting tool 22 within the guide opening 44 and with the depth stop abutting against the guide surface 30 may result in an interior channel 40 forming an osteotomy within the bone 16 along the interior cutting profile 38. In this way, the cutting guide 12 may provide for the accurate formation of the interior channel 40 aligned with the blind depth of the interior feature 36.

Referring back to FIG. 1, the exemplary cutting guide 12 may incorporate two parallel guide plates 14 that define a guide opening 44 through which the longitudinal axis A_{L} of the cutting tool 22 may extend. In various implementations, the cutting tool 22 may correspond to a rotary, reciprocating, and/or sagittal cutting tool. In the example shown, the cutting tool 22 may correspond to a surgical drill, burr, or endmill; that may be configured to rotate about the longitudinal axis A_{L}, such that a shaft 46 is aligned with the interior channel 40 along the first guide plate 14a and the second guide plate 14b, which are arranged in parallel along the guide opening 44. In this configuration, the shaft 44 of the cutting tool 22 may closely fit (e.g., provide a clearance fit) within parallel interior guide surfaces 48 formed by the guide plates 14. In this way, a cutting trajectory of the longitudinal axis A_{L} and corresponding cutting path associated with the rotation R of the cutting tool 22 may be limited to conform to a guide plane 50 of the interior guide surfaces 48.

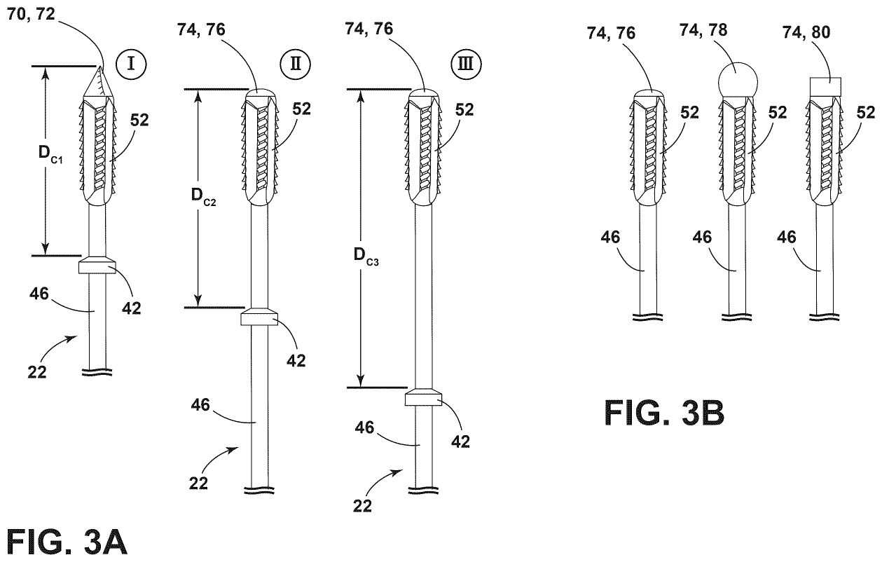

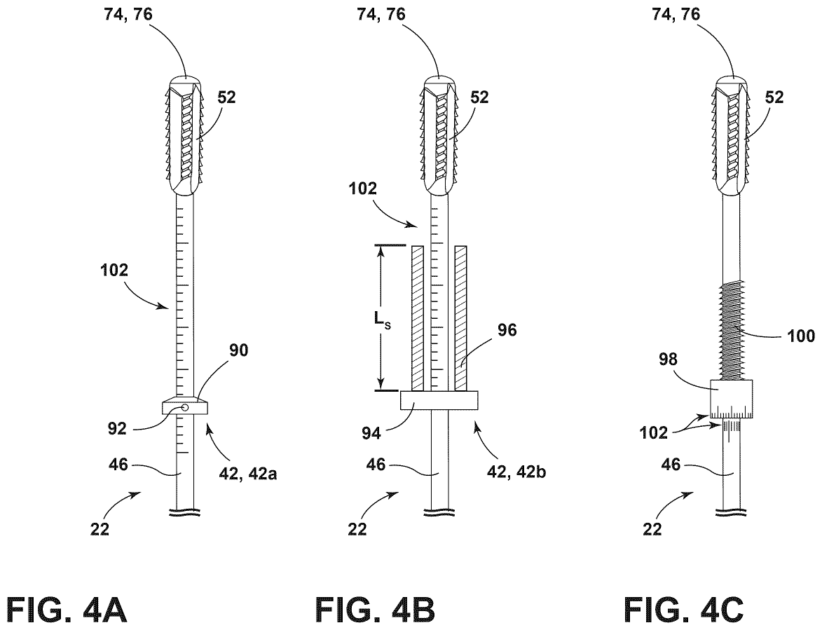

Still referring to FIGS. 1 and 2, the engagement of the cutting tool 22 within the guide opening 44 of the cutting guide 12 may further be restricted by the engagement of the depth stop 42 with the guide profile 28 formed by the guide surface 30. As discussed in further detail in reference to FIGS. 3A, 4A, 4B, and 4C; the depth stop 42 may be adjusted such that the cutting depth D_{C} or a plunge depth extending from the guide surface 30 may be adjusted to the target depth D_{T} or one or more intermediate cutting depths therebetween. As a result of the operation of the cutting tool 22 in coordination with the cutting guide 12, the constant cutting depth D_{C} of a cutting head 52 of the cutting tool 22 may result in the interior channel 40 or osteotomy formed through the bone 16 along a cutting path 54 extending along the guide plane 50 and with a constant cutting depth DC defined by the engagement of the depth stop 42 with the guide surface 30. In this way, a blind depth of the interior channel may conform to the guide profile 28 in alignment with the interior feature 36.

As best shown in FIG. 2A, the guide profile 28 may correspond to a feature or characteristic formed within the bone 16. In the example shown, the interior feature 36 may correspond to an interior transition of the bone that may correspond to an endocortical surface 56 extending between the cancellous bone 58 and the cortical bone 60. Accordingly, the interior feature 36 along which the guide profile 28 extends may correspond to a transition from a first hardness to a second hardness of the bone that may be defined along a blind transition depth within the bone 16. As best demonstrated in FIG. 1, the controlled removal of the bone 16 along the cutting path 54 would be completely blind without implementing the cutting guide 12. By implementing the cutting guide 12, the depth stop 42 may engage the guide surface 30, such that the cutting depth D_{C} of the interior channel 40 conforms to guide profile 28. In this way, the cutting tool 22 may engage the bone 16 to accurately create the interior channel 40 with a depth corresponding to the guide profile 28 without uncertainty. In the example of FIGS. 1 and 2, the cutting path 54 (?) is aligned with the guide plane 50. However, it shall be understood that, in various implementations, the guide plates 14 may be contoured such that the guide opening 44 forms a contoured cutting path 54 through which the shaft 46 of the cutting tool 22 may be guided for a variety of operations.

As best demonstrated in FIG. 2C, shaft 46 of cutting tool 22 may extend through the guide opening 44, such that the shaft 46 is aligned with the guide plane 50 along the first and second guide plates 14a, 14b. The cutting depth D_{C} is set as the length of the cutting tool 22 extending from the distal tip of the cutting head 52 back to the depth stop 42 along the longitudinal axis. In this configuration, the engagement of the depth stop 42 with the guide surface 30 may result in the interior channel 40 being made along the target depth D_{T} and in conformance with the guide profile 28. As previously discussed, the guide profile 28 may be defined based on one or more interior features or characteristics of the bone 16 that may be identified by one or more scanning techniques. In this way, the guide profile 28 and the corresponding blind depth of the cutting tool 22 along the cutting path 54 may be achieved by traversing the cutting tool 22 along the cutting path 54 with the depth stop 42 in contact with the guide surface 30.

As discussed herein, the interior feature or characteristic 36 of the bone 16 may be identified and mapped to determine the guide profile 28 based on one or more cross-sectional imaging techniques. For example, cross-sectional imaging techniques may include computed tomography (CT scans), magnetic resonance imaging (MRI), and various similar forms of imaging and computed tomography capable of capturing and depicting cross-sectional characteristics of the anatomy of a patient. Accordingly, the guide profile 28 may be identified based on one or more preoperative images or scans aligned with the guide plane 50 associated with the region targeted for the osteotomy or interior channel 40 formed through the bone 16. Once aligned, the guide profile 28 may be offset over the thickness of the bone 16 and along a depth of the guide plates 14 along the guide surface 30. In this configuration, the opposing positive surface profile or contour associated with the interior feature 36 of the bone 16 may be mapped to form the guide surface 30 and provide for accurate formation of the interior channel 40 at the target depth D_{T} along the cutting path 54.

In addition to the alignment of the cutting tool 22 with the cutting path 54 and the guide profile 28, the cutting apparatus 10 may further incorporate additional features that may improve the accuracy and procedural results from the osteotomy or various orthopedic procedures. Referring now to FIG. 3A, a plurality of compatible cutting tools 22 are shown including a plurality of different cutting heads 52. In operation, the cutting tools 22 and corresponding cutting heads 52 may be implemented sequentially to gradually form the interior channel at the target depth D_{T} over a plurality of predetermined cutting depths D_{C}.

Though the specific number of cutting tools 22 and corresponding cutting heads 52 may vary, the example of FIG. 3A demonstrates a first cutting tool 22a with a first cutting head 52a and a second cutting tool 22b with a second cutting head 52b. The first cutting head 52a may correspond to a piercing tip 70 comprising a tapered or sharpened distal point 72 and a first head diameter Φ₁. The piercing tip 70 of the first cutting tool 22a may assist or facilitate the removal of the dense cortical bone 60 as well as assist in plunging through the more porous cancellous bone 58. Accordingly, the piercing tip 70 of the first cutting head 52 may provide for an aggressive rough cut forming the interior channel 40 at the first head diameter Φ₁. As denoted by I, the piercing tip 70 may be implemented at a first cutting depth D_{C1} and may be applied primarily to form the interior channel 40 through the proximal cortical bone 58 along the first side or first surface 24a of bone 16.

As further demonstrated in the sequential steps denoted as II and III, the second cutting tool 22b having the second cutting head 52b may include a dull or blunt distal tip 74. The blunt tip 74 may include a convex distal end 76, as shown, or may similarly include a flattened or concave distal end. The blunt tip 74 may limit occurrences of rapid plunging along the cutting depth D_{C} when cutting through soft cancellous bone 58. As shown in steps II and III, the cutting depth may be sequentially increased when forming the interior channel 40 from the first cutting depth D_{C1} to increasing depths of a second cutting depth D_{C2} and a third cutting depth D_{C3}. In this way, the cutting depth DC may be gradually increased to the target depth D_{T} to ensure that the remaining structure of the bone 16 along the second side or second surface 24b beyond the distal tip of the cutting head 52 may preserve the corresponding bone structure associated with the guide profile 28. As is apparent in FIG. 3A, the position of the depth stop 42 along the longitudinal axis of the cutting tool 22 may be sequentially increased or adjusted to provide the controlled, preset or predetermined cutting depths D_{C}. Further details regarding the structure and assembly of the depth stop 42 and the corresponding engagement with the shaft 46 of the cutting tool 22 are demonstrated and discussed in reference to FIGS. 4A-4C.

Referring now to FIG. 3B, variations of the blunt tip 74 are shown that may allow the cutting apparatus 10 to be modified or adjusted to suit various preferences and/or procedures. As shown, a first blunt tip 74a may include the convex distal end 76, as demonstrated in FIG. 3A. Additionally, a second blunt tip 74b may include a bulbous, spherical distal end 78 that may prevent the cutting tool 22 from plunging or increasing the cutting depth DC. The bulbous, spherical distal end 78 of the second blunt tip 74b may be formed of a flexible or malleable (e.g., polymeric) material that may be compressed to conform to the dimensions or width of the interior channel 40 as formed by the piercing tip 70. As further demonstrated in FIG. 3B, a third blunt tip 74c may comprise a flattened, rectangular distal end 80. In implementations incorporating the blunt tip 74, particularly the spherical distal end 78 and the rectangular distal end 80, the blunt tip 74 may provide for a cutting offset for offset depth d that may prevent increasing the cutting depth D_{C} via plunging operations while also clearing the side walls of the interior channel 40. Accordingly, the cutting tool 22 of the surgical cutting apparatus 10 may be implemented in a variety of ways to suit various procedures and user preferences.

Referring now to FIGS. 4A-4C, examples of the depth stops 42 are shown that may be implemented with the cutting apparatus 10. As shown in FIG. 4A, a first depth stop 42 may correspond to a stop collar 90 that may be fixed in position along the shaft 46 via a set screw 92 or pin. As shown in FIG. 4B, a second depth stop 42b may comprise a fixed stop collar 94 with a removable sleeve 96. In contrast with the stop collar 90 that may be adjusted along the length of the shaft 46, the fixed stop collar 94 may vary the cutting depth D_{C} by providing multiple removable sleeves 96 having different sleeve lengths L_{S}. Accordingly, the cutting depth D_{C} may be adjusted by interchanging the removable sleeves 96 having different sleeve lengths L_{S}.

As demonstrated in FIG. 4C, a third depth stop 42 may incorporate a threaded stop collar 98. The threaded stop collar 98 may engage a corresponding threaded portion 100 of the shaft 46, allowing the cutting depth D_{C} to be adjusted by rotating the threaded stop collar 98 about the longitudinal axis A_{L}. In each of the depicted implementations, the exemplary depth stops 42 may include graduations 102 or depth markings that may indicate the cutting depth D_{C} corresponding to the position of the depth stop 42. In the example of FIG. 4C, the threaded stop collar 98 may be calibrated based on the pitch of the threads or similarly based on the threads per unit distance of the threaded portion 100, such that the distance traveled by the threaded stop collar 98 may be calibrated per rotation of the threaded stop collar 98 about the longitudinal axis. The graduations 102 depicted in FIG. 4C are denoted about the circumference of the shaft 46 and the circumference of the threaded stop collar 98. Accordingly, the depth stop 42 may be implemented in a variety of ways, such that the cutting apparatus 10 may be flexibly implemented.

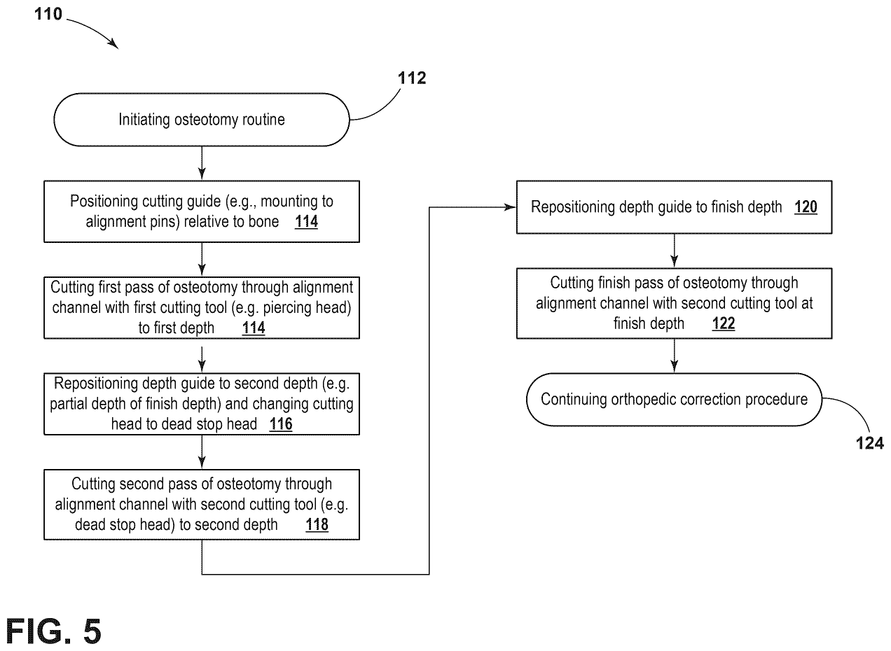

Referring now to FIG. 5, the surgical apparatus 10 may be applied to complete various orthopedic procedures. In particular, FIG. 5 demonstrates a method 110 for performing an osteotomy. The method may begin by initiating the osteotomy routine 110, including various tissue preparation steps that may reveal the first surface 24a of the bone 16 (112). Once the tissue is prepared and the bone 16 is exposed, the cutting guide 12 may be positioned (e.g., via the alignment features 18), such that the guide plane 50 and the corresponding cutting path 54 are aligned with the anatomy through which the interior channel 40 is to be formed (114). Once the cutting guide 12 is positioned relative to the bone 16, the interior channel 40 may be formed at the first cutting depth D_{C1} with the first cutting tool 22a (116). As previously discussed, the first cutting tool 22a may comprise the first cutting head 52a having the piercing tip 70 that may be suited to plunging the cutting tool 22 through the hard exterior cortical bone 60. Following completion of the first cutting pass of step 114, the interior channel 40 may be formed along the cutting path 54 at a first rough depth.

Following the first pass with the piercing tip 70 of the first cutting tool 22a, the cutting tool 22 may be changed to the second cutting tool 22b having the blunt tip 74 and a position of the depth stop 42 may be adjusted to increase the cutting depth to the second cutting depth D_{C2} (116). With the second cutting tool 22b, the method 110 may continue by cutting the interior channel 40 to the second cutting depth D_{C2}, which may approach the target depth D_{T} along the guide profile 28 (118). In steps 120 and 122, the position of the depth stop 42 along the shaft 46 may be adjusted and a finished cutting pass may be executed, thereby forming the interior channel 40 along the cutting path 54 and having the third cutting depth D_{C3} aligned with the target depth D_{T} along the guide profile 28. Following completion of the formation of the interior channel 40 or osteotomy of the bone 16, the orthopedic correction procedure may continue in step 124.

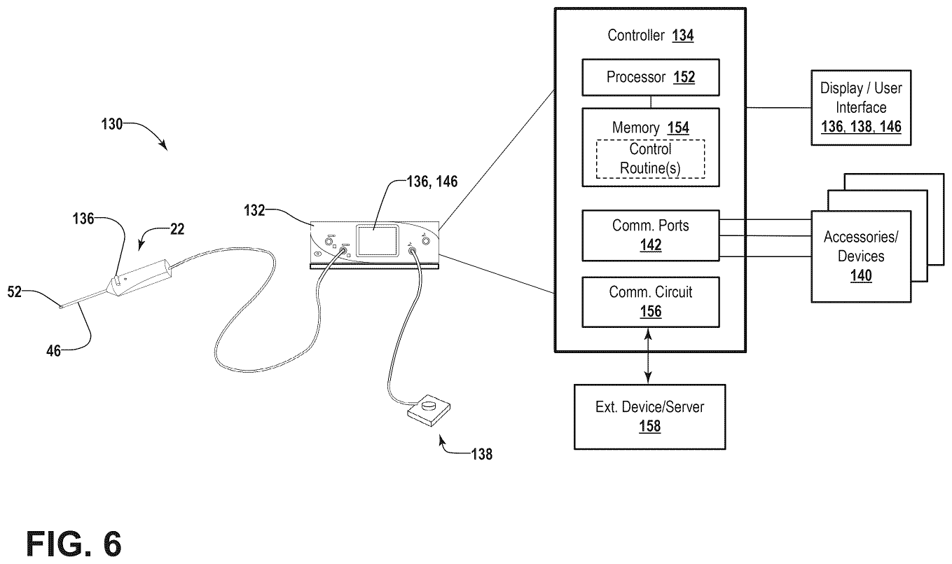

Referring now to FIG. 6, a control system 130 is shown demonstrating the control console 132 comprising a controller 134 of the cutting tool 22. In operation, the controller 134 may receive inputs via a control interface or user interface 136, which may be incorporated on the handpiece 22a of the cutting tool 22 or provided via one or more peripheral control accessories 138. In various examples, the operation of the system 130 is discussed in reference to the operation of the cutting tool 22. However, it shall be understood that the operation of the system 130 may commonly provide for the concurrent use and control of two or more surgical devices 140, which may include various handpieces, peripheral devices, remote controls, and various other devices or accessories that may be beneficial in a medical or surgical environment. For example, the surgical devices 140 may include various laser or radio frequency cutting or operating utilities in the form of ablation devices, catheters, pumps, suction or aspiration devices, and similar tools that may be in communication with the control console 132 via a plurality of communication ports 142 or various other communication connection (e.g., a device network).

As discussed throughout the disclosure, the system 130 may provide for coordinated communication and control of the cutting tool 22, which may correspond to a rotary surgical instrument, sagittal cutting instrument, or various cutting tools that may include distal cutting heads or blades. In operation, the controller 134 may determine or access information defining the model, style, dimensions, operating ranges, etc. for the cutting head 52 and the cutting tool 22. Such model or accessory information may be programmed into, accessed by, or otherwise identified by the controller 134 for each of the configurations of the cutting head 52. For example, the cutting style, features, operating parameters (e.g., speed, duration, etc.) may be identified in response to a manual programming input, selection in an accessory library or database, and/or detected by the controller 134 based on information accessed from the cutting head 52. In some cases, identifying style, model, dimensional, operating speed ranges and limits, usage restrictions (time limits, cutting pass limits, etc.), manufacturer information, usage statistics and various forms of information related to the cutting head 52 may be accessed by the controller 134. Such information may be accessed via an electronic identification circuit or tag (e.g., radio frequency identification [RFID]) incorporated in the cutting head 52 or the cutting tool 22. The electronic identification circuit may be accessed by the controller 134 by one or more communication circuits 144 or communication ports 142. In this way, the controller 134 may update the operation of the cutting tool 22 in response to the specific style, dimensions, and operating configurations of each of the interchangeable cutting heads 52.

In some implementations, the system 130 may include one or more display screens 146 that communicate with various controllers and surgical devices 140 similar to those discussed herein. As previously discussed, the control console 132 may be in communication with one or more surgical devices 140 or accessories 138 that may be associated with the operation of the control console 132. For example, the accessories 138 may correspond to one or more electronic or electromechanical buttons, triggers or pedals (e.g., pressure sensitive or single actuation foot pedals), and additional devices communicatively connected to the communication ports 142. The display screen 146/user interface 136 of the control console 132 may include one or more switches, buttons, dials, and/or displays, which may include soft-key or touchscreen devices incorporated in a display (e.g., liquid crystal display [LCD], light emitting diode [LED] display, cathode ray tube [CRT], etc.). In response to inputs received from the display screen 146 and/or user interface 136, the controller 134 may activate or adjust the settings of the control signals communicated to the cutting tool 22. The control signals generated by the console controller 134 may be configured for operation in response to the selected operating configuration, routine, duty cycle, etc. The output signals communicated from the communication port 142 to the surgical apparatus 10 may be generated by various signal generators, motor controllers, or power supplies that may provide for operation of power electronic operations (e.g., motor drive signals and supply current) based on the instructions, commands, or signals communicated from a processor 152 of the controller 134 for the associated operating configuration. Accordingly, the console controller 134 may be operable to generate signals to drive or control the motion, rotation, activation, intensity, and various other operating characteristics of the surgical apparatus 10.

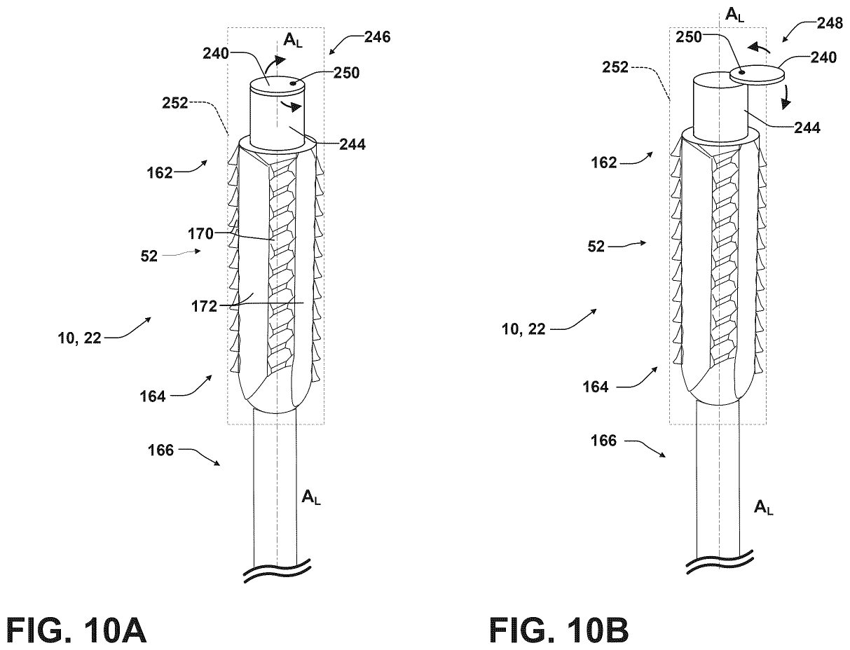

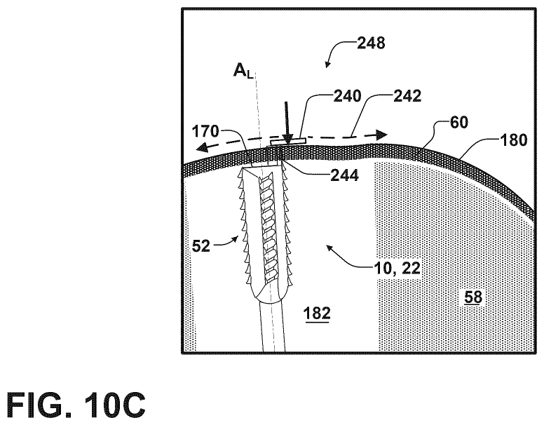

The processor(s) 152 of the controller 134 may be implemented as one or more microprocessors, microcontrollers, application-specific integrated circuits (ASIC), or other circuitry configured to perform instructions, computations, and control various input/output signals to control the control system 130. The instructions and/or control routines of the system 130 may be accessed by the processor(s) 152 via a memory 154. The memory 154 may comprise random access memory (RAM), read only memory (ROM), flash memory, hard disk storage, solid state drive memory, etc. Each of the processor(s) 152 and memory devices 134 may be implemented to suit the corresponding functionality or sophistication of the surgical apparatus 10 or cutting tool and the corresponding control requirements of the controller 134. The controller 134 may incorporate additional communication circuits or input/output circuitry, generally represented in FIG. 10 as the communication circuit(s) 156, which may be implemented to communicate with one or more peripherals, devices, remote computers or servers 158, etc. The communication circuit(s) 156 may complement or support the operating capability of the communication ports 142. In general, the communication circuit 156 may provide for communication via a variety of communication protocols to support operation of the surgical apparatus 10. In an exemplary embodiment, the circuitry associated with the communication ports 142 may include digital-to-analog converters, analog-to-digital converters, digital inputs and outputs, as well as one or more communication interfaces or buses. The circuits associated with the communication ports 142 and/or the communication circuit 156 may be implemented with various communication protocols, such as serial communication (e.g., CAN bus, I2C, etc.), parallel communication, or network communication (e.g., RS232, RS485, Ethernet). In some cases, the communication circuit 156 may also provide for wireless network communication (Wi-Fi, Bluetooth®, Ultra-wideband [UWB], etc.). In some examples, the controller 134 may be in communication with one or more of the external devices 158 (e.g., control devices, peripherals, servers, etc.) via the communication circuit 156. Accordingly, the control console 132 may provide for communication with various devices to update, maintain, and control the operation of the system 130.

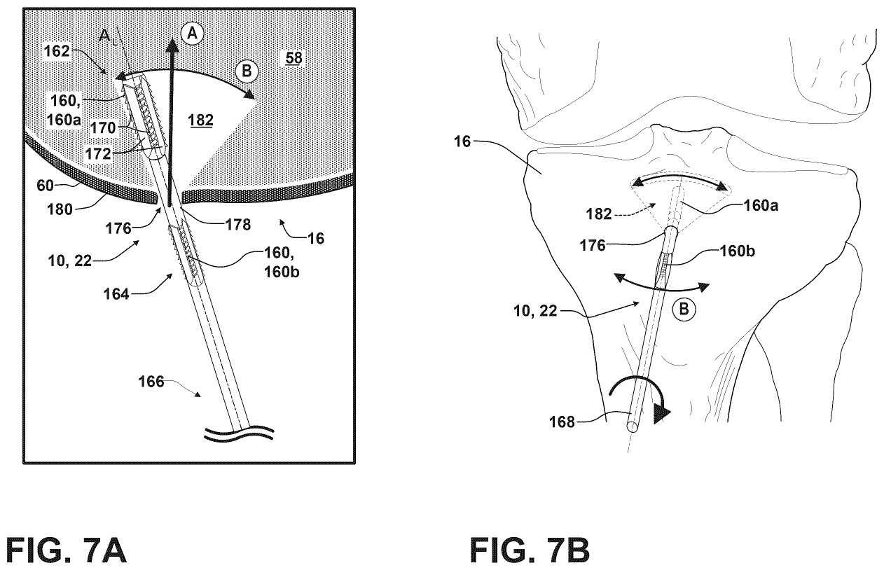

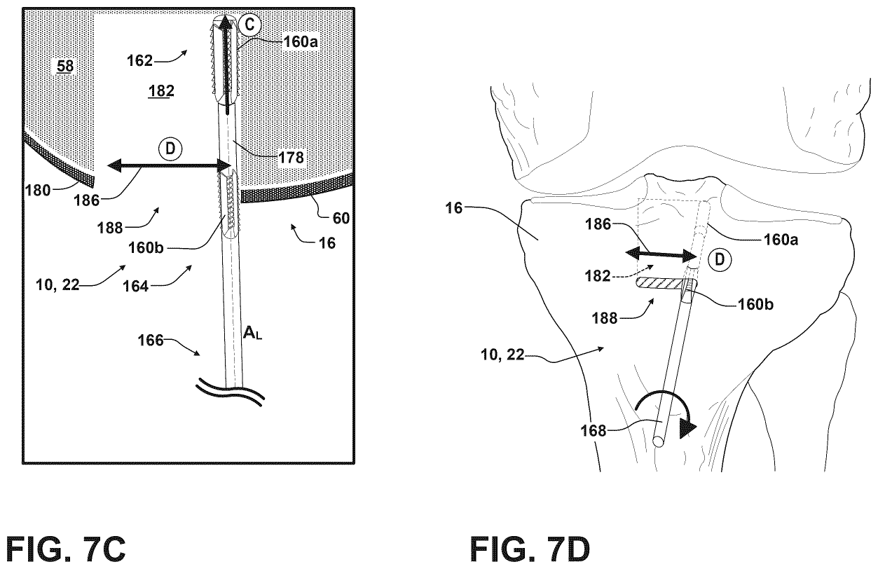

Referring now to FIGS. 7A, 7B, 7C, and 7D, an implementation of the cutting tool 22 is shown comprising a plurality of cutting heads 160 positioned in a spaced-apart configuration along a length of the longitudinal axis Aₗ. In the example shown, a first cutting head 160a is positioned at a distal end 162 and a second cutting head 160b is positioned along an intermediate portion 164 positioned between the distal end 162 and a proximal end portion 166. In various implementations, the proximal end portion 166 may correspond to a tool interface or shank 168 configured to be engaged by a rotary tool. Each of the cutting heads 160 may correspond to milling/drilling heads and/or burrs comprising one or more cutting edges 170 that may be radially spaced and separated by corresponding flutes 172 or channels. In this configuration, each of the cutting heads 160 may engage the bone 16 to effectuate various cutting or resection procedures.

As demonstrated in FIGS. 7A and 7B, an exemplary cutting method for the cutting tool 22 is demonstrated. In operation, a first opening 176 may be formed by plunging the first cutting head 160a into the bone 16 through the cortical bone 60 along the longitudinal axis A_{L}as demonstrated in annotated Operation A. As shown, the cutting tool 22 may be plunged into the bone 16 along a longitudinal axis A_{L} until a radially smooth or cylindrical portion 178 is aligned with the cortical bone 60 and/or an exterior surface 180 of the first opening 176. Once aligned within the first opening 176, the cylindrical portion 178 may be pivoted against the surfaces of the cortical bone 60 forming the first opening 176 allowing the first cutting head 160a to cut an interior cavity 182 within the cancellous bone 58 as shown in Operation B. This operation may utilize the comparatively hard or rigid structure of the cortical bone 60 to support the pivoting of the cutting tool. By utilizing the surfaces of the cortical bone 60 forming the first opening 176 as a fulcrum, the pivoting of the cutting tool 22 may be achieved with a high level of precision to remove the cancellous bone 58 and support a variety of procedures.

As further demonstrated in FIGS. 7C and 7D, the cutting tool 22 may further be implemented to extend or enlarge the first opening 176 along a translational cutting path 186 with the second cutting head 160b. As denoted by Operation C, the cutting tool 22 may be plunged further to a second depth into the bone 16 until the second cutting head 160b is aligned with the first opening 176 or, more generally, with the cortical bone 60 as demonstrated in FIG. 7C. Once aligned, the cutting tool 22 may be translated along the translational cutting path 186 as demonstrated by Operation D and the associated arrow. As shown, the translational cutting path 186 may extend substantially parallel to the surface 180 to form an elongated slot or opening through the cortical bone 60 and the remaining cancellous bone 58. The cutting path 186 may be aligned or constrained to the cutting path 54 and/or the guide profile 28 as previously discussed in reference to FIGS. 1 and 2A-2C. In this way, the first opening 176 may be extended or enlarged to form a second opening 188 as well as extend the corresponding proportions of the interior cavity 182. Accordingly, the cutting apparatus 10 provided by the disclosure may be implemented in a variety of ways to suit various procedures.

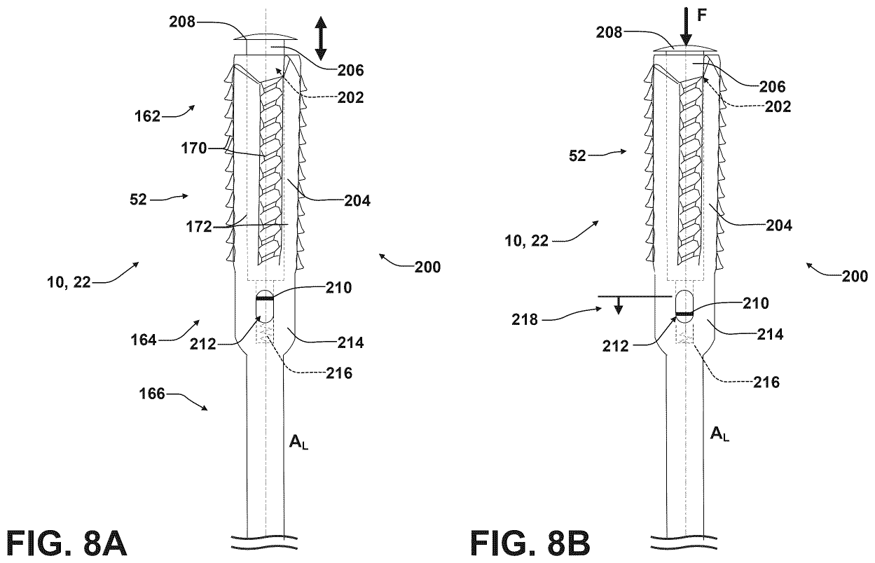

Referring now to FIGS. 8A and 8B, in some implementations, the cutting apparatus 10 may incorporate a force-detection gauge 200 that may be implemented to provide feedback for a user identifying the pressure applied to the distal end 162 of the cutting tool 22 along longitudinal axis A_{L}. Like other implementations of the cutting tool 22, the example demonstrated in FIGS. 8A and 8B may comprise the shaft 46 extending from the proximal end portion 166 to the distal end portion 162 along the intermediate portion 164. In the example shown, the force-detection gauge 200 may comprise an interior passage 202 formed within a body 204 of the cutting tool 22. An interior shaft 206 or rod may be disposed in the interior passage 202 and comprise a distal protrusion 208 protruding from the distal end 162 of the interior passage 202. Additionally, the interior shaft 206 may extend through the interior passage 202 from the distal end portion 162 to the intermediate portion 164 of the body 204. In this configuration, the proximal end portion 166 of the interior shaft 206 may comprise an indicator 210 disposed on an indicator surface of the interior shaft 206 that is aligned with a viewing aperture 212 formed through a side wall 214 of the cutting tool 22 and aligned with the interior passage 202. As demonstrated in FIGS. 8A and 8B, the indicator may translate along the longitudinal axis A_{L} over a length of the viewing aperture 212 indicating a force F applied to the distal protrusion 208. In this configuration, the position of the indicator 210 within the viewing aperture 212 may be representative of the force or pressure applied along the longitudinal axis A_{L} and corresponding force applied back to the bone 16 or tissue of a patient.

In various implementations, the displacement of the interior shaft 206 within the interior passage 202 may be controlled by an opposing spring force applied by a spring 216 disposed within the interior passage 202. In this configuration, the spring 216 may deflect in response to the translation of the interior shaft 206 along the longitudinal axis A_{L}. The deflection resulting from the force F may result in an opposing spring force applied by the spring 216 that may be calibrated to correspond to a maximum procedural force or force range desired for one or more procedures. In this configuration, a translational position 218 of the indicator 210 may be representative of the incrementally increasing force F associated with the operating pressure applied to the cutting tool 22 along the longitudinal axis A_{L}. Accordingly, the indicator 210 may provide for a force or pressure indication associated with the operation of the cutting tool 22 to improve operator feedback.

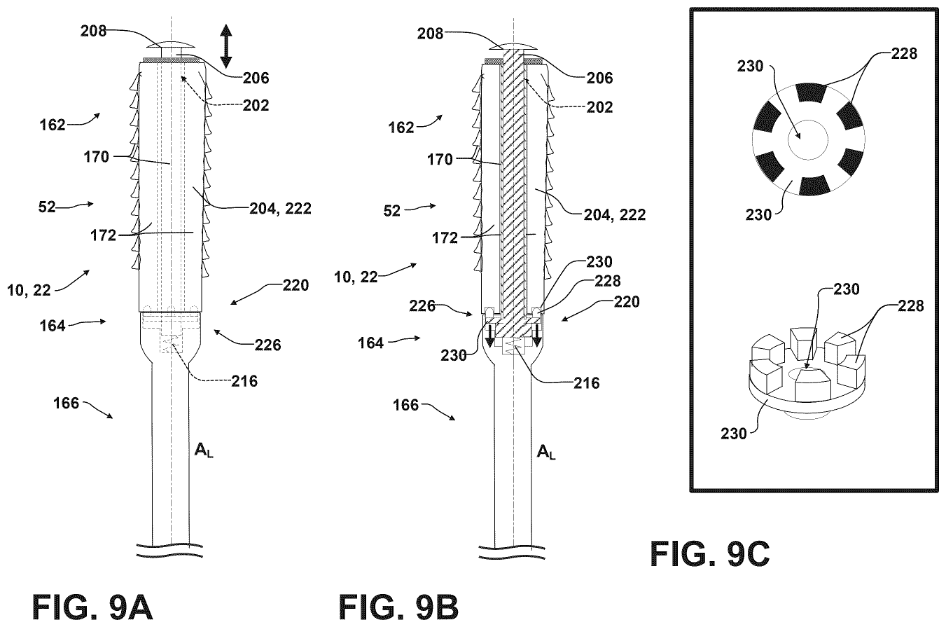

Referring now to FIGS. 9A, 9B, and 9C; in some implementations, the cutting apparatus 10 may comprise a release feature 220 that may be configured to release the cutting head 52 from the shank 168. For example, in operation, the distal protrusion 208 may engage the shaft 206 as similarly described in reference to FIGS. 8A and 8B. As best demonstrated in FIG. 9B, the external force applied to the distal protrusion 208 to the shaft 206 may cause the spring 216 to compress within a barrel 222 forming the interior passage 202 along a length of the cutting head 52. In response to the external force, the spring 216 may compress allowing a mating interface 226 of the release feature 220 to withdraw from the barrel 222 along the longitudinal axis A_{L}. As illustrated in FIG. 9C, the mating interface 226 may comprise a plurality of mating protrusions 228 that may engage corresponding receiving apertures 230 formed along approximal end portions of the barrel 222. In this configuration, the translation of the shaft 206 through the interior passage 202 may result in the withdrawal of the mating protrusions 228 from the receiving apertures 230, allowing the barrel 222 and the cutting head 52 to spin freely relative to the shaft 206 and the shank 168. In this way, the drive to the cutting apparatus 10 may be disengaged in response to the force or pressure applied to the distal end 162 exceeding a predetermined force necessary to compress the spring 216.

In the example shown, the mating interface 226 is formed by a compression disk 232 from which the mating protrusions 228 extend parallel to the shaft 206. The compression disk 232 may comprise a shaft bore 234 that may be configured to receive the shaft 206 and interconnect to the shaft 206 and the shank 168 via a keyed interface or similar coupling interface. The mating protrusions 228 may correspond to beveled teeth or posts that may engage the receiving apertures 230 of the barrel 222 along the longitudinal axis. Though demonstrated in the example shown with the mating protrusions 228 extending from the compression disk 232, it shall be understood that the protrusions 228 and apertures 230 may be interchangeably implemented or applied in combination in connection with the barrel 222 of the cutting head 52 and/or the surface of the compression disk 232.

Referring now to FIGS. 10A, 10B, and 10C, in some implementations, the cutting apparatus 10 may comprise a distal stop 240 or withdrawal guide feature that may allow the path of the cutting edge 52 to conform to a positive exterior cutting profile 222 of the bone 16 aligned with the distal end 162 of the cutting tool 22. In various implementations, one or more of the cutting edges 172 may extend along a distal protrusion 244 to the distal stop 240. As shown, the distal stop 240 may correspond to a rotating plate that may be selectively positioned between a stowed position 246 and a deployed position 248. As demonstrated in FIG. 10A, the distal stop 22 may be rotated about a connection interface 250 to align with a longitudinal cutting envelope 252 of the cutting tool 22 spaced about the longitudinal axis A_{L}. As shown in FIG. 10B, in the deployed position 248, the distal stop 240 may be positioned outside the longitudinal cutting envelope 252 of the cutting tool 22. In this configuration, the distal stop 240 may constrain the translation of the cutting tool 22 along the longitudinal axis A_{L} as a result of the distal stop 240 engaging a proximally positioned exterior surface 180 of the bone 16 as demonstrated and further discussed in reference to FIG. 10C.

As shown in the example of FIG. 10C, the distal stop 240 is configured in the deployed position 248 such that the movement or translation of the cutting tool 22 proximally along the longitudinal axis A_{L} is limited to conform to the positive exterior cutting profile 222 of the bone 16. In this configuration, the translation of the cutting tool 22 through the tissue or bone 16 may be limited to correspond to the distally located exterior surface 180 of the bone 16. Further, upon completion of the associated resection implemented with the guidance of the distal stop 240, the distal stop 240 may be positioned in the stowed position 246 and withdrawn from the bone 16 or tissue. Though discussed in reference to specific examples, each of the features and operating procedures described in reference to the cutting apparatus 10 may be implemented alone or in combination to suit the needs of a particular procedure or preferences of a user. Accordingly, the surgical cutting apparatus 10 of the disclosure may be flexibly implemented in a wide variety of applications.

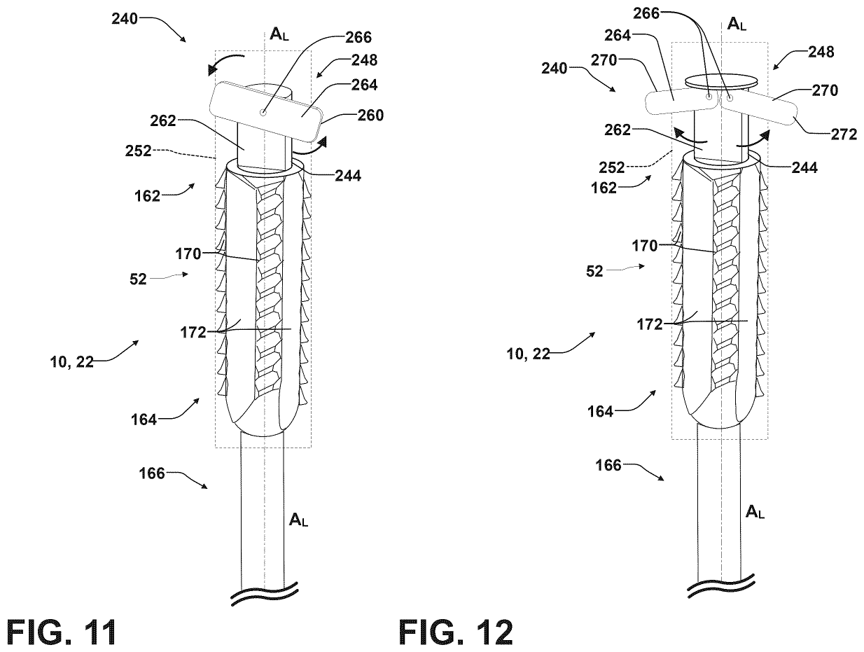

Referring now to FIGS. 11 and 12, additional examples of distal stops 240 are demonstrated. As shown in FIG. 11, the distal stop 240 may be implemented as a rotating, retraction plate 260. As shown, the retraction plate 260 may engage a flat 262 or channel formed through the distal protrusion 244. The retraction plate 260 may correspond to a rectangular or otherwise elongated body 264 that may rotate relative to the longitudinal axis and the distal protrusion 244 about a pivotal connection 266. In operation, the proportions of the elongated body 264 may rotate relative to the longitudinal axis and protrude beyond the cutting envelope 262 and corresponding bore formed by the cutting head 52 through the tissue or bone associated with the procedure. The rotation of the retraction plate 260 may be induced based on the rotation of the cutting head 52 and the corresponding centrifugal force acting on an imbalanced weight of the opposing sides 268 of the elongated body 264. In this way, the rotation of the cutting head 52 may result in the rotation of the retraction plate 260, such that the elongated dimensions of the elongated body 264 extend beyond the cutting envelope 252, thereby preventing the cutting apparatus 10 from retracting from the exterior surface 180 as previously discussed in reference to FIG. 10C.

Referring now to FIG. 12, yet another example of the distal stop 240 is shown. In some implementations, a plurality of retraction plates 270 may be interconnected with the distal protrusion 244 extending from the cutting head 52. Similar to the retraction plate 260, the plurality of retraction plates 270 may be interconnected to the flat 262 or similar interface surfaces or channels formed in the distal protrusion 244 via pivotal connections 266. In operation, the rotation of the cutting head 52 may result in centrifugal force acting on a distal portion 272 of the retraction plates 270 opposing the pivotal connection 266. In this configuration, the retraction plates 270 may extend on opposing sides of the cutting head 52 outside the cutting envelope 252 while the cutting head is spinning rapidly. The extension of the retraction plates 270 outside the cutting envelope 252 may prevent the retraction of the cutting apparatus 10 from a cavity formed through the tissue (e.g., bone) of a patient. In this way, the distal stop 240 may provide for the cutting apparatus 10 to be guided along the exterior surface 180 of the bone along the exterior cutting profile.

According to some aspects of the disclosure, a surgical cutting apparatus comprises a cutting tool including a cutting head extending along a longitudinal axis to an acting end portion and a cutting guide including an engagement surface configured to engage a first side of a bone. The cutting guide forms an opening extending from a guide surface, wherein the guide surface forms a guide contour offset from and aligned with a positive contour of a second side of the bone. A depth guide is in connection with the cutting tool and defines a cutting depth along the longitudinal axis, wherein the depth guide engages the guide surface along the opening and the cutting depth is adjusted along the opening by the engagement of the depth guide with the guide contour.

According to various aspects, the disclosure may implement one or more of the following features or configurations in various combinations:
the guide contour of the guide surface defines a depth of cut of the cutting tool extending through the opening;
the second side of the bone opposes the first side of the bone;
the guide contour extends along the positive contour formed by a cortex of the second side of the bone;
the guide contour is aligned with an endocortical surface of the second side of the bone;
the passage forms an elongated slot extending along a cutting path of the cutting tool along the first side of the bone;
the guide contour follows the positive contour of the second side of the bone along the cutting path;
   the guide contour forms a depth guide aligned with a blind depth profile formed within a cancellous portion of the bone along the cutting path;
the cutting head is a rotary cutting head;
the cutting head comprises one of a burr, a drill, and an endmill;
a depth stop in connection with a rotary shaft of the cutting tool, wherein a position of the depth stop along the longitudinal axis sets a cutting depth of the cutting head relative to the guide surface; and/or
the depth stop engages the guide surface and limits the cutting depth to the positive contour of the second side of the bone.

According to another aspect of the disclosure, a cutting guide for orthopedic procedures comprises a body comprising an engagement surface configured to position the cutting guide on a first side of a bone and an opening extending through the body from a guide surface. The opening forms a passage configured to receive a cutting tool, wherein the guide surface forms a guide contour offset from and aligned with a positive contour of a second side of the bone.

According to various aspects, the disclosure may implement one or more of the following features or configurations in various combinations:
the guide contour of the guide surface defines a depth of cut of the cutting tool extending through the opening, the second side of the bone generally opposes the first side of the bone;
the guide contour extends along the positive contour formed by an interior cortex of the second side of the bone;
the positive contour corresponds to an endocortical surface of the second side of the bone and the guide contour is offset from the positive contour on the first side of the bone opposite the second side;
the positive contour extends within the bone between a compact bone portion and a spongy bone portion;
the passage forms an elongated slot extending along a cutting path of the cutting tool along the first side of the bone;
the guide contour follows the positive contour of the second side of the bone along the cutting path; and/or
the guide contour forms a depth stop surface aligned with a blind depth profile formed along a perimeter of a cancellous portion of the bone along the cutting path.

According to yet another aspect of the disclosure, a method for performing an osteotomy comprises positioning a cutting guide on a first side of a bone; aligning at least one cutting tool within an opening of the cutting guide, the opening comprising at least one guide translational surface forming a lateral cutting path through the bone; and engaging the cutting tool with the first side of the bone and cutting the bone to a guide depth of the osteotomy, wherein the cutting comprises engaging a depth stop of the cutting tool in contact a guide contour of the cutting guide, wherein the guide depth of the guide contour is aligned a transition depth of an interior transition of the bone along the cutting path.

According to various aspects, the disclosure may implement one or more of the following features or configurations in various combinations:
the transition depth of the bone is defined by the interior transition of the bone from a first hardness to a second hardness;
the transition of the bone from the first hardness to the second hardness is defined by a transition from cancellous bone to cortical bone proximate to the second side of the bone;
the at least one cutting tool comprises a plurality of cutting tools comprising a first cutting tool comprising a sharpened or piercing tip and a second cutting tool comprising a dull or blunt head;
the cutting through the first side of the bone comprises first cutting a channel in the bone to a first depth with the sharpened or piercing tip and extending the channel to a second depth with the dull or blunt head; and/or
aligning the depth guide with the transition depth along a longitudinal axis of the cutting tool, wherein the second depth is extended to the transition depth by engaging the depth stop of the cutting tool along the guide contour at the transition depth.

According to some aspects of the disclosure, a cutting apparatus for orthopedic procedures comprises a rotary cutting tool forming a body extending along a longitudinal axis and extending from a proximal end comprising a tool interface to a distal end; a first cutting head at the distal end; and a second cutting head positioned along an intermediate portion of the body between the proximal end and the distal end. The first cutting head and the second cutting head are separated by a shaft portion.

According to various aspects, the disclosure may implement the following feature or configuration in various combinations:
the shaft portion comprises a smooth exterior surface evenly spaced about the longitudinal axis.

According to another aspect of the disclosure, a method for implementing a cutting apparatus for an orthopedic procedure is provided. The method comprises forming a first opening through a cortical bone with a first cutting head of the cutting apparatus; plunging the first cutting head into a cancellous bone to a first depth through the first opening; and pivoting a smooth intermediate portion of the cutting apparatus against the cortical bone forming the first opening, thereby forming an interior cavity within the cancellous bone.

According to various aspects, the disclosure may implement one or more of the following features or configurations in various combinations:
engagement of the smooth intermediate portion with the cortical bone forming the first opening provides a fulcrum against which the cutting apparatus pivots along a length;
plunging the first cutting head to a second depth deeper within the cancellous bone, thereby engaging a second cutting head, proximal of the smooth intermediate portion, within the first opening; and/or
translating the second cutting head through the cortical bone enlarging the first opening to a second opening.

According to yet another aspect of the disclosure, a cutting apparatus for orthopedic procedures comprises a rotary cutting tool forming a body extending along a longitudinal axis and extending from a proximal end comprising a tool interface to a distal end forming a cutting head, wherein the body forms an interior passage extending from the distal end to an intermediate portion of the body. A shaft disposed in the interior passage extends from a distal protrusion protruding from the distal end to a proximal portion comprising an indicator surface. A viewing aperture extends through a side wall of the body from the interior passage, wherein an indicator on the indicator surface translates along the longitudinal axis along the viewing aperture indicating a force applied to the distal protrusion.

According to various aspects, the disclosure may implement one or more of the following features or configurations in various combinations:
a spring disposed in the interior passage, wherein the spring deflects in response to a translation of the shaft along the longitudinal axis;
the spring applies a spring force to the shaft equivalent to a maximum procedural force in response to a proximal translation of the indicator within the viewing aperture;
a spring force of the spring is calibrated to a force range for at least one procedure associated with the operation of the rotary cutting tool; and/or
the viewing aperture is positioned along the intermediate portion and the intermediate portion is proximal of the cutting head.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

The claims:

1. A surgical cutting apparatus comprising:
a cutting tool comprising a cutting head extending along a longitudinal axis to an acting end portion;
a cutting guide comprising an engagement surface configured to engage a first side of a bone, the cutting guide forming an opening extending from a guide surface, wherein the guide surface forms a guide contour offset from and aligned with a positive contour of a second side of the bone; and
a depth guide in connection with the cutting tool and defining a cutting depth along the longitudinal axis, wherein the depth guide engages the guide surface along the opening and the cutting depth is adjusted along the opening by the engagement of the depth guide with the guide contour.

2. The surgical cutting apparatus according to claim 1, wherein the guide contour of the guide surface defines a depth of cut of the cutting tool extending through the opening.

3. The surgical cutting apparatus according to claim 1, wherein the second side of the bone opposes the first side of the bone.

4. The surgical cutting apparatus according to claim 1, wherein the guide contour extends along the positive contour formed by a cortex of the second side of the bone.

5. The surgical cutting apparatus according to claim 4, wherein the guide contour is aligned with an endocortical surface of the second side of the bone.

6. The surgical cutting apparatus according to claim 1, wherein the passage forms an elongated slot extending along a cutting path of the cutting tool along the first side of the bone.

7. The surgical cutting apparatus according to claim 6, wherein the guide contour follows the positive contour of the second side of the bone along the cutting path.

8. The surgical cutting apparatus according to claim 1, wherein the guide contour forms a depth guide aligned with a blind depth profile formed within a cancellous portion of the bone along the cutting path.

9. The surgical cutting apparatus according to claim 1, wherein the cutting head is a rotary cutting head.

10. The surgical cutting apparatus according to claim 9, wherein the cutting head comprises one of a burr, a drill, and an endmill.

11. The surgical cutting apparatus according to claim 1, further comprising:
a depth stop in connection with a rotary shaft of the cutting tool, wherein a position of the depth stop along the longitudinal axis sets a cutting depth of the cutting head relative to the guide surface.

12. The surgical cutting apparatus according to claim 11, wherein the depth stop engages the guide surface and limits the cutting depth to the positive contour of the second side of the bone.

13. A cutting guide for orthopedic procedures comprising:
a body comprising an engagement surface configured to position the cutting guide on a first side of a bone; and
an opening extending through the body from a guide surface, the opening forming a passage configured to receive a cutting tool, wherein the guide surface forms a guide contour offset from and aligned with a positive contour of a second side of the bone.

14. The cutting guide according to claim 13, wherein the guide contour of the guide surface defines a depth of cut of the cutting tool extending through the opening.

15. The cutting guide according to claim 13, wherein the guide contour extends along the positive contour formed by an interior cortex of the second side of the bone.

16. The cutting guide according to claim 13, wherein the positive contour corresponds to an endocortical surface of the second side of the bone and the guide contour is offset from the positive contour on the first side of the bone opposite the second side.

17. The cutting guide according to claim 13, wherein the positive contour extends within the bone between a compact bone portion and a spongy bone portion.

18. The cutting guide according to claim 13, wherein the passage forms an elongated slot extending along a cutting path of the cutting tool along the first side of the bone.

19. The cutting guide according to claim 18, wherein the guide contour follows the positive contour of the second side of the bone along the cutting path, and wherein the guide contour forms a depth stop surface aligned with a blind depth profile formed along a perimeter of a cancellous portion of the bone along the cutting path.

20. A method for performing an osteotomy comprising:
positioning a cutting guide on a first side of a bone;
aligning at least one cutting tool within an opening of the cutting guide, the opening comprising at least one guide translational surface forming a lateral cutting path through the bone; and
engaging the cutting tool with the first side of the bone and cutting the bone to a guide depth of the osteotomy, wherein the cutting comprises engaging a depth stop of the cutting tool in contact a guide contour of the cutting guide, wherein the guide depth of the guide contour is aligned at a transition depth of an interior transition of the bone along the cutting path.
